(19) United States
(12) Patent Application Publication
(10) Pub. No.: US 2004/0058436 A1
(43) Pub. Date: Mar. 25, 2004

(54) Cell-detaching reactor for scaled-up inoculation of anchorage-dependent cell culture

(75) Inventors: Yuanxing Zhang, Shanghai (CN); Xiangming Sun, Shanghai (CN); Weiming Fan, Shanghai (CN); Jian Lu, Shanghai (CN)

Correspondence Address: MERCHANT & GOULD PC, P.O. BOX 2903, MINNEAPOLIS, MN, 55402-0903, US

(73) Assignee: East China University of Science and Technology, Shanghai (CN)

(21) Appl. No.: 10/455,136

(22) Filed: Jun. 5, 2003

(30) Foreign Application Priority Data
Sep. 20, 2002 (CN) 02137069.9

(51) Int. Cl. C12M 1/02

(52) U.S. Cl. 435/295.1

(57) ABSTRACT

A cell-detaching reactor is provided to prepare single cell suspension for inoculation of anchorage-dependent cells between the scaled-up bioreactors, which is especially useful in a commercial-scale process. The cell-detaching reactor of the invention comprises a trypsinizing zone and a separating zone, which are separated by a screen with mesh size between the diameters of the cells and the carriers.

### BACKGROUND OF THE INVENTION

1. Technical Field of the Invention

The invention relates to a cell-detaching reactor used for scaled-up inoculation anchorage-dependent cells from a small bioreactor to a large one.

2. Description of Related Art

The anchorage-dependent cell lines, for example, the animal cells, have been extensively cultured to produce recombinant proteins, recombinant virus and viral vaccines. These cells must be grown on the matrix surface. Microcarriers such as Cytodex 1, 2 and 3, manufactured by Pharmacia Co. (Sweden) are preferred for the culture of anchorage-dependent cells. While the animal cells are cultured commercially on microcarriers in bioreactors under agitation, a series of bioreactors gradually expanding in size are needed to prepare a great quantity of seed cells for the final culture in a large bioreactor in the production scale. Therefore, the inoculation among bioreactors seems to be determinant for the performance of the whole cycle of the culture.

Three kinds of inoculation methods have been reported so far:

1. Digestion inoculation A large number of T-flasks or roller bottles are used to prepare seed cells. Upon cells growing to confluence, trypsin solution is added into these T-flasks or roller bottles. Then the cells are collected as seed cells and transferred into a bioreactor (Wentz and Schugerl, Enzyme Microb. Technol. 14:68-75 (1992)).

This method is limited to a small-scale cell culture (generally, not more than 3 liters in volume). Also, it is laborious and susceptible to contamination, which makes it difficult to be scaled up to a commercial application, especially with the anchorage-dependent cells.

2. In situ digesting inoculation Upon the cells grow to confluence in a seed bioreactor, the agitation is stopped, and the supernatant is carefully drawn off after the microcarriers settle to the bottom completely. Then, the sediment is washed with buffer solution (generally, phosphate buffer solution, PBS). Then, the buffer solution is carefully drawn off before adding the trypsin solution. After the trypsinization is completed, the culture medium containing high concentration of serum is added into the bioreactor to block the trypsin activity. Then, the microcarriers with the attached cells are recovered into a sterile receptacle through a vibrating screen under moderate vibration to detach the cells harmlessly. The obtained cell suspension is transferred as seed cells to the subsequent bioreactor (Wilktor et al., U.S. Pat. No. 4,664,912)

Obviously, this method is very cumbersome. Each step comprises a delay before the completion of sedimentation of the microcarrier, which eventually prolongs the operation time, and, moreover, a loss of cells. Further more, since it is difficult to get rid of the solutions completely in each step, the undesired trypsin residue in the inoculum suspension may not only decrease the performance of the serum proteins, but also adversely influence the growth of cells in subsequent bioreactors.

3. Bead-to-bead inoculation When the cells on microcarriers in the seed bioreactor have grown to a desired density, the culture is directly transferred into the subsequent bioreactor containing new microcarriers. The cells on the old microcarriers will gradually transfer to the new ones. This is the so called “bead-to-bead” inoculation (Hu et al., Cytotechnology 33:13-19 (2000), Cong et al., Biotechnol. Lett. 23:881-885 (2001)).

Because the velocity and the efficiency of the transfer from the old carriers to the new ones are very low, most of the cells are still attached to the old microcarriers but grow at a very low rate due to the contact inhibition in the new reactor. Even there may be, sometimes, no cells transferring to and growing on the new microcarriers. Moreover, the growth of the cells on the microcarriers is inhomogeneous. The ones on the new microcarriers may be in the exponential phase, while those on the old microcarriers may be in the steady phase Thus, it is difficult to control the monolayer convergence time of the cell growth, which finally results in a low cell density and low productivity. Therefore, the direct inoculation, though simple, is not the most desirable. (Sun et al., J. East China Univ. Sci. Technol. 25:567-569 (1999), Sun et al., J. East China Univ. Sci. Technol. 25:570-573 (1999)).

Therefore, there is still a need for a more efficient method of inoculation of anchorage-dependent cells between scaled-up bioreactores, by which a single cell suspension of seeds may be obtained with high validity and recovery.

### SUMMARY OF THE INVENTION

The object of the present invention is to provide a cell-detaching reactor for the inoculation of anchorage-dependent cells between the scaled-up bioreactors.

The object of the invention is fulfilled by providing a cell-detaching reactor for the inoculation of anchorage-dependent cells between the scaled-up bioreactors, comprising:

(I) a trypsinizing zone consisting of a cylinder, wherein the said trypsinizing zone comprises:

(i) a cover hermetically affixed to the top of the cylinder,

(ii) an agitating device hermetically installed in a rotatable manner through the center of the said cover,

(iii) at least one feed inlet hermetically connected to the cover in a fluid interconnection, and

(iv) at least one gas outlet/inlet hermetically connected to the cover in a fluid interconnection; and

(II) a separating zone consisting of the bottom that is hermetically connected to the lower edge of the trypsinizing zone, wherein the said separating zone comprises:

(i) a steel screen fixed to the upper edge of the bottom, the mesh size of which is between the diameters of the anchorage-dependent cells and the microcarriers,

(ii) at least one inlet/outlet for the medium, at least one inlet for the wash solution/trypsin solution and at least one outlet for the wash solution/trypsin solution, each being hermetically connected to the bottom in a fluid interconnection.

### DETAILED DESCRIPTION OF THE INVENTION

Specifically, the main body of the trypsinizing zone in the invention is a cylinder, which is equivalent to or slightly larger than the seed reactor in volume. The ratio of height to diameter of the said cylinder is about 0.5˜1.5. The said cylinder may be made of stainless steel or glass.

The said cover is hermetically affixed to the top of the cylinder. The said cover may be made of stainless steel. In the center of the cover, there is a hole through which extends the pivot of the agitation device. Along the peripheral part distal to the said center are distributed the said feed inlet(s) for introducing the culture from the previous seed bioreactor, and the said gas outlet(s)/inlet(s) for introducing the sterile gas to control the pressure and the atmosphere in the said cylinder. The exit of the gas pipe(s) in the cylinder must be above the liquid level therein. All the said inlets and outlets are hermetically connected to the said cover in a fluid interconnection. For this purpose, many means and methods are available and well-known in the art. In one embodiment of the invention, the seal between the said cylinder and the said cover is accomplished by placing an O-ring in the cover flange and secure the upper edge of the cylinder in the seal groove in the O-ring. Surrounding the cylinder, there may be several fixing bolts vertically extending from the bottom to the cover symmetrically distributed in order to press the said cover, cylinder and bottom to form strict seals among each other.

The agitating device of the invention may be, for example, anchor impeller agitator, arrow-shaped paddle, disk agitator, pitched turbine agitator and crew propeller agitator, which may be arranged in monolayer or multilayers. The diameter of the paddle of the agitator is about 30˜95% of the inner diameter of the said cylinder. The lowest paddle is about 3˜50 millimeters vertically distant from the upper surface of the screen. The hermetic and rotatable attachment between the agitating device and the cover may be accomplished via, e.g., mechanical pivot gland or magnetic transmission sealing techniques.

The main body of the said separating zone is the said bottom that is hermetically connected to the said cylinder. The said bottom may be of any type commonly used in the bioreactors. Also, it may be modified into a conical shape. The said bottom may be made of stainless steel. It is hermetically connected to the cylinder in usual ways.

In one embodiment of the invention, the seal between the said cylinder and the said bottom is accomplished by placing an O-ring in the bottom and secure the lower edge of the cylinder in the seal groove in the O-ring. Surrounding the cylinder, there may be several fixing bolts vertically extending from the bottom to the cover symmetrically distributed in order to press the said cover, cylinder and bottom to form strict seals among each other.

The screen of the invention may be tightly affixed to the fixing ring by pressing, adhering or welding. Then, the said fixing ring is placed in the bottom flange so that the screen and the flange of the bottom is in the same surface, see FIG. 2. The said screen may be selected from the commercially available types usually made of stainless steel or nylon. The mesh size of screen must be larger than the diameter of the cells and smaller than the diameter of the microcarriers, in order to retain the carriers with/without cells on the screen while permitting the detached cells to pass through. Thereby, after the trypsinization and before the separation of the cells from the carriers, the trypsin solution can be completely removed, leaving the carriers with the attached cells retained in the trypsinizing zone, which advantageously prevents damaging the cells caused by the trypsin residue. Moreover, during the said separation, the mesh size as said above permits the passage of the free cells through the screen into the bottom, but not the carriers. Thereby, a single cell suspension is obtained in the bottom that are then discharged and transferred to the subsequent bioreactor. Given the cell line and microcarrier, informations regarding the diameters of both the cells and the carriers are quite accessible to the skilled in the art, which makes the determination of the mesh size quite easy. In a preferred embodiment of the invention, the surface of the screen is siliconized in order to prevent the microcarriers from adhering to the screen to block the mesh and finally decrease the filtering efficiency. The siliconization may be carried out by using, for example, trimethyl chlore silane, dichlorodimethylsilane (Davis et al. (ed.), Basic Methods in Molecular Biology, Prentice-Hall International Inc (1994)) or hexamethyldisilane (Ezheng (ed.), Tissue Culture and Molecular Cytotechnology, Beijing press, (1995)).

There are at least one inlet/outlet for the medium, at least one inlet for the wash solution/trypsin solution and at least one outlet for the wash solution/trypsin solution hermetically connected to the bottom in a fluid interconnection. The said inlet(s)/outlet(s) for the medium are used to introduce medium to make single cell suspension after trypsinization and, later, drain off the said suspension to be further transferred into the subsequent bioreactor for culture.

The said inlet(s) for the wash solution/trypsin solution and outlet(s) for the wash solution/trypsin solution are controlled, as desired, by the valves in the connected pipelines. The said wash solution or the said trypsin solution is introduced through the reactor of the invention down to up, and drained off through the outlet on the bottom of the reactor after the treatment with either.

In an embodiment of the invention, after the completion of the incubation in the previous seed bioreactor, the culture containing the seed cells attached to the microcarriers is introduced into the said trypsinizing zone of the said cell-detaching reactor through the feed inlet on the cover. Sterile gas at appropriate pressure (0.01˜0.15 MPa constant pressure) is introduced into the reactor through the gas inlet to raise the inner pressure of the reactor to dischage the supernatant through medium inlet/outlet at the bottom under pressure. Alternatively, a pump, such as a peristaltic pump, may be used to drain off the said supernatant through the said medium inlet/outlet. Additionally, other drainage methods well-known in the art can also be used alone or in combination. Then, the wash solution is introduced into the cell-detaching reactor from the bottom through the said inlet(s) for wash solution/trypsin solution. After wash, the wash solution is discharged through the said outlet(s) for the wash solution/trypsin solution. Then, trypsin solution is introduced into the cell-detaching reactor from the bottom through the said inlet(s) for wash solution/trypsin solution. During the trypsinization, agitator works at such a low speed that the trypsinization is carried out evenly throughout the said trypsinizing zone, while maintaining the cells attached to the microcarriers. After the trypsinization is completed, the trypsin solution is completely discharged through the outlet(s) for the wash solution/trypsin solution at the bottom. Then, the medium is introduced into the cell-detaching reactor through medium inlet/outlet at the bottom, and the agitation is switched to a high speed to detach/separate the cells from the microcarriers. Then, the resultant single cell suspension are discharged through the medium inlet/outlet at the bottom and, then, transferred into the subsequent bioreactor.

The cell-detaching reactor of the invention can advantageously change the way of inoculation, significantly improve the culture efficiency and permit the scale-up of the anchorage-dependent cell culture. The cell-detaching reactor of the invention may be widely used in various applications including, for example, commercially culturing anchorage-dependent cells such as CHO, BHK, Vero cells, etc, to produce recombinant proteins, viral vaccines and recombinant virus for gene therapy.

Further scope of applicability of the present invention will become apparent from the detailed description given hereinafter. However, it should be understood that the detailed description and specific examples, while indicating preferred embodiments of the invention, are given by way of illustration only, since various changes and modifications within the spirit of the invention will become apparent to those skilled in the art from this detailed description.

### BRIEF DESCRIPTION OF THE DRAWINGS

The present invention will become more fully understood from the detailed description given hereinbellow and the accompanying drawings which are given by way of illustrations only, and thus are not limitative of the present invention, wherein,

FIG. 1 shows an embodiment of the cell-detaching reactor of the invention;

FIG. 2 shows the attachment and fixation of the screen in the cell-detaching reactor of the invention;

FIG. 3 shows the flows of the materials in a typical operation of the cell-detaching reactor of the invention;

FIG. 4 shows the growth profiles of Vero cells inoculated form a 1-liter seeds reactor using the cell-detaching reactor of the invention into a 5-liter culture reactor.

### EXAMPLES

### Example 1

### Inoculation of CHO Cells

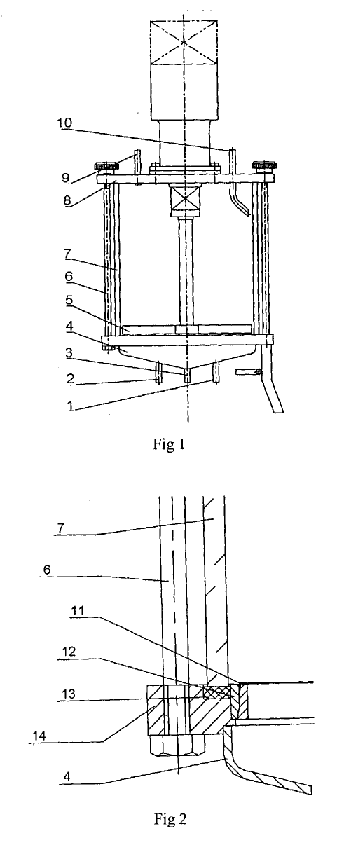

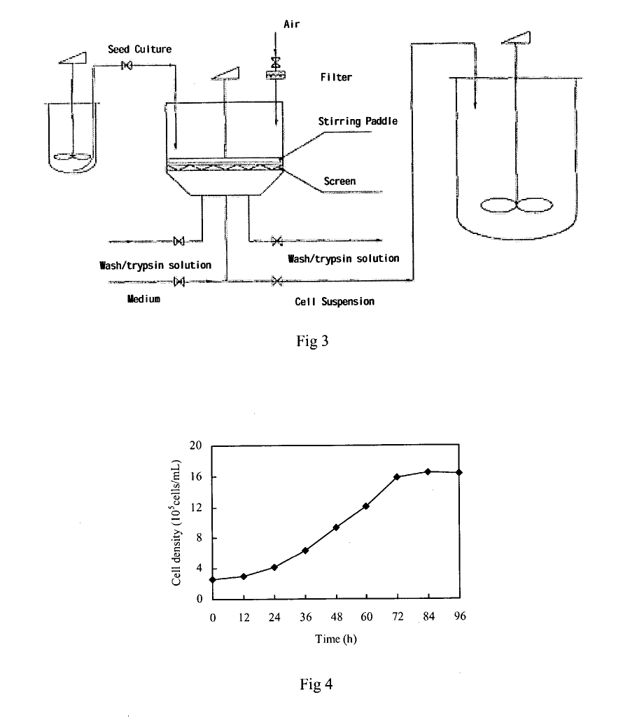

To culture the CHO cells, the cell-detaching reactor of the invention is utilized to conduct the inoculation from a 1-liter reactor to a 5-liter reactor. The 1-liter reactor has a work volume of about 0.7 liter and is used for microcarrier based culture. The 5-liter reactor has a work volume of about 3.5-liter and is a packed-bed based bioreactor. The structure of the cell-detaching reactor is shown in FIG. 1, and the flow of the materials therein is shown in FIG. 3. The volume of the cell-detaching reactor is 3.5 liter. As shown in the FIGS. 1-2, the cylinder 7 made of glass is 186 mm in height and 143 mm in diameter. The cover 8 is made of stainless steel. The agitator 5 is installed through the central hole (not shown) in the cover, and hermetically and rotatably affixed to the cover using the mechanical pivot gland. The O-ring (not shown) is fixed in cover 8. Four open holes for bolts 6 are distributed evenly along the periphery of the flange of the cover. As said above, the cover and the cylinder are hermetically connected by the flange, O-ring and fixing bolts 6. The agitator 5 in this example is a monolayer of cambered stirring paddles, having a diameter of 95% of that of the cylinder. The lower surface of the paddle is 50 millimeters distant from the upper surface of screen. The stirring paddle is affixed to the pivot of a driving motor by fixing screws (not shown). The bottom 4 is conical and made of stainless steel. The seal ring 13 is fixed in the seal ring cavity 13 in the bottom. Four open holes for the bolts 6 are distributed evenly along the periphery of the flange 14 of the bottom. As said above, the bottom and the cylinder are hermetically connected by the flange, O-ring and fixing bolts 6. The fixed ring 12 on which the screen is fixed is placed in the bottom. The screen 11 is a 200 mesh screen made of 316L stainless steel. It is known that the average diameter of CHO cells is 15 μm and the microcarrier Cytodex-1, 180 μm. Thus, the mesh size of screen 11 is determined to be 60 μm. The screen is further siliconized with 2% trimethylchlorosilane in chloroform.

Initially, phosphate buffer solution is introduced into the cell-detaching reactor. After autoclaved at 121° C. for 30 min, the cell-detaching reactor is connected, under sterile condition, to the 1L seed bioreactor via the feed inlet 10 on the cover and to the 5-L culture bioreactor via the medium inlet/outlet 3. When the cell density in the 1-L seed bioreactor reaches 10×10⁶ cells per milliliter, the culture is pressed into the trypsinizing region of the cell-detaching reactor through the feed inlet 10 by sterile gas. Then, sterile gas at constant pressure of 0.1 MPa is introduced into the cell-detaching reactor through gas inlet/outlet 9. Thus, the supernatant of the culture is completely discharged through medium inlet/outlet 3 on the bottom under increased inner pressure. The microcarriers with attached cells are retained on the screen.

Wash solution preheated to 37° C. is introduced through inlet 1 for the wash solution/trypsin solution on the bottom. Wash is conducted for about 1 min under agitation at 20 rpm. The inner pressure is increased again as said above to completely press out the wash solution through outlet 2 for the wash solution/trypsin solution. Then, the trypsin solution preheated to 37° C. is introduced through inlet 1 for the wash solution/tyrosine solution on the bottom. Trypsinization is conducted for about 6 min under agitation at 20 rpm. The inner pressure is increased again as said above to completely press out the trypsin solution through outlet 2 for the wash solution/trypsin solution. Then, the culture medium preheated to 37° C. is introduced into the cell-detaching reactor through medium inlet/outlet 3 on the bottom, and agitated at 120 rpm for 6 minutes to detach the cells from the microcarriers. The inner pressure is increased again, as said above, to completely press the resultant single cell suspension out of the cell-detaching reactor through the medium inlet/outlet 3. The said suspension is then transferred into the subsequent 5-liter culture bioreactor at the seeding cell density of 2×10⁵ cells/ml for further perfusion culture. After culturing for 11 days, the final cell density in the 5L packed-bed bioreactor is 1.2×10⁷ cells/ml.

### Example 2

### Inoculation of Vero Cells

To culture the Vero cells, the cell-detaching reactor of the invention are utilized to conduct the inoculation from a 1-liter reactor to a 5-liter reactor. The 1-liter reactor has a work volume of about 0.7 and the 5-liter reactor, 3.5-liter. Both reactors are used for microcarrier based culture. The structure of the cell-detaching reactor is shown in FIG. 1, and the flow of the materials therein is shown in FIG. 3. The volume of the cell-detaching reactor is 3.5 liter. As shown in FIGS. 1-2, cylinder 7 made of glass is 186 mm in height and 143 mm in diameter. The cover 8 is made of stainless steel. The agitator 5 is installed through a central hole (not shown) in the cover, and hermetically and rotatablly affixed to the cover using the mechanical pivot gland. The O-ring (not shown) is fixed in the cover 8. Four open holes for the bolts 6 are distributed evenly along the periphery of the flange of the cover. As said above, the cover and the cylinder are hermetically connected by the flange, the O-ring and the fixing bolts 6. The agitator 5 in this example is a monolayer of cambered stirring paddles, having a diameter of 30% of that of the cylinder. The lower surface of the paddle is 3 millimeters distant from the upper surface of the screen. The stirring paddle is affixed to the pivot of a driving motor by fixing screws (not shown). The bottom 4 is conical and made of stainless steel. The seal ring 13 is fixed in the seal ring cavity 13 in the bottom. Four open holes for the bolts 6 are distributed evenly along the periphery of the flange 14 of the bottom. As said above, the bottom and the cylinder are hermetically connected by the flange, O-ring and fixing bolts 6. The fixed ring 12 on which the screen is fixed is placed in the bottom. The screen 11 is a 200 mesh screen made of 316L stainless steel. It is known that the average diameter of CHO cells is 15 μm and the microcarrier Cytodex-1, 180 μm. Thus, the mesh size of the screen 11 is determined to be 60 μm. The screen is further siliconized with 2% trimethylchlorosilane in chloroform.

Initially, phosphate buffer solution is introduced into the cell-detaching reactor. After autoclaved at 121° C. for 30 min, the cell-detaching reactor is connected, under sterile condition, to the 1 L seed bioreactor via the feed inlet 10 on the cover and to the 5-L culture bioreactor via the medium inlet/outlet 3. When the cell density in the 1-L seed bioreactor reaches 1.3×10⁶ cells per milliliter, the culture is pressed into the trypsinizing region of the cell-detaching reactor through the feed inlet 10 by sterile gas. Then, sterile gas at constant pressure of 0.1 MPa is introduced into the cell-detaching reactor through gas inlet/outlet 9. Thus, the supernatant of the culture is completely discharged through medium inlet/outlet 3 on the bottom under the increased inner pressure. The microcarriers with attached cells are retained on the screen.

Wash solution preheated to 37° C. is introduced through the inlet 1 for the wash solution/trypsin solution on the bottom. Wash is conducted for about 1 min under agitation at 20 rpm. The inner pressure is increased again as said above to completely press out the wash solution through the outlet 2 for the wash solution/trypsin solution. Then, the trypsin solution preheated to 37° C. is introduced through the inlet 1 for the wash solution/tyrosine solution on the bottom. Trypsinization is conducted for about 6 min under agitation at 20 rpm. The inner pressure is increased again as said above to completely press out the trypsin solution through the outlet 2 for the wash solution/trypsin solution. Then, the culture medium preheated to 37° C. is introduced into the cell-detaching reactor through the medium inlet/outlet 3 on the bottom, and agitated at 120 rpm for 6 minutes to detach the cells from the microcarriers. Inner pressure is increased again, as said above, to completely press out the resultant single cell suspension out of the cell-detaching reactor through the medium inlet/outlet 3. The said suspension is then transferred into the subsequent 5-liter culture bioreactor at the seeding cell density of 2.6×10⁵ cells/ml for further perfusion culture. After culturing for 4 days, the final cell density in the SL bioreactor is 1.6×10⁶ cells/ml. The growth profile of Vero cells cultured in the 5-liter reactor is shown in FIG. 4.

What is claimed:

1. A cell-detaching reactor for the inoculation of anchorage-dependent cells between the scaled-up bioreactors, comprising:
(I) a trypsinizing zone consisting of a cylinder, wherein the said trypsinizing zone comprises:
(i) a cover hermetically affixed to the top of the cylinder,
(ii) an agitating device hermetically installed in a rotatable manner through the center of the said cover,
(iii) at least one feed inlet hermetically connected to the cover in a fluid interconnection, and
(iv) at least one gas outlet/inlet hermetically connected to the cover in a fluid interconnection; and
(II) a separating zone consisting of the bottom that is hermetically connected to the lower edge of the trypsinizing zone, wherein the said separating zone comprises:
(i) a steel screen fixed to the upper edge of the bottom, the mesh size of which is between the diameters of the anchorage-dependent cells and the microcarriers,
(ii) at least one inlet/outlet for the medium, at least one inlet for the wash solution/trypsin solution and at least one outlet for the wash solution/trypsin solution, each being hermetically connected to the bottom in a fluid interconnection.

2. The cell-detaching reactor of claim 1, wherein, the said screen is siliconized.

3. The cell-detaching reactor of claim 1, wherein, the diameter of the paddle of the said agitating devices is 30˜95% of the inner diameter of the said cylinder.

4. The cell-detaching reactor of claim 1, wherein, the said agitating device has cambered paddle.

5. The cell-detaching reactor of claim 1, wherein, the said agitating device has monolayer or multilayer of paddles, and the lowest edge of the paddles is 3˜50 millimeters distant from the upper surface of the said screen.
